(19) United States
(12) Patent Application Publication
(10) Pub. No.: US 2014/0119504 A1
(43) Pub. Date: May 1, 2014

(54) Radiography apparatus

(71) Applicant: Lodovico Taddia, Medicina (IT)

(72) Inventor: Lodovico Taddia, Medicina (IT)

(21) Appl. No.: 13/660,682

(22) Filed: Oct. 25, 2012

(51) Int. Cl. A61B 6/00

(52) U.S. Cl. CPC A61B 6/502
USPC 378/37

(57) ABSTRACT

A radiography apparatus for mammography comprises a supporting column or stand having a main direction of development, a ground supporting base to which the column is secured and a radiography assembly; the radiography assembly, secured in a sliding manner to said column and moving along the same between a first operational position and a second operational position, comprises an emitter of rays for radiography, a corresponding detector for acquisition of the image obtained by means of said rays and a system for positioning and maintaining in position a breast to be X-rayed, operationally active between said emitter and detector; the column moves with respect to the base between a first raised operational position and a second lowered operational position.

The present invention concerns a radiography apparatus and in particular a radiography apparatus for breast radiography.

### BACKGROUND OF THE INVENTION

In general, radiography apparatus for breast radiography refers to both mammography and radiography apparatus for tomosynthesis.

A radiography apparatus for mammography of known type, which will be explicitly referred to below without loss of generality, basically comprises a supporting column, also known as “stand” in the jargon of the sector, to which a radiography assembly is secured in a sliding manner, moving vertically up and down the column, for example by means of a slider.

The radiography apparatus is provided with a ground supporting base to which the stand is secured.

The radiography assembly comprises an X ray emitter and a corresponding detector for acquisition of the image obtained by means of the rays emitted.

The radiography assembly furthermore comprises, arranged between the emitter and the detector, a breast support element and a corresponding element for compression of the same, necessary for correct positioning of the breast during the radiography. The radiography assembly is commonly secured to the stand by means of a horizontal arm around which the assembly can rotate.

In this way radiography can be performed from different angles (in general at least 2) so that the body to be examined is not in the shade, hidden or transparent to the radiography rays.

A big drawback of said types of apparatus derives from the fact that they cannot be effectively used in the performance of biopsies since in these operations the breast must be preferably arranged in a pendulous position.

It should be noted that the radiography apparatus for biopsies is substantially analogous to the one previously described. The biggest differences are that the biopsy apparatus has a substantially horizontal development so that the patient can be positioned above it with the breast hanging down and that in the radiography assembly the breast compression elements are integral with the detector while the emitter is mobile with respect to the latter.

Generally, therefore, hospitals often have two different types of machine, one with substantially vertical development for mammography and a second one for the performance of breast biopsies, allowing the breast to be arranged in a pendulous position, said position entailing, among other things, the downward movement of any suspicious foreign body during the radiography.

In the use of two different machines, a lesion identified by mammography may not be locatable during the biopsy.

The necessary presence of two different machines furthermore involves higher costs for equipping an efficient laboratory and increased maintenance costs.

In this context, the main technical aim of the present invention is to propose a radiography apparatus that overcomes the above-mentioned drawbacks.

### SUMMARY OF THE INVENTION

One object of the present invention is to propose a radiography apparatus that allows radiography to be performed also on a breast arranged in a pendulous position. A further object of the present invention is to propose a radiography apparatus that allows reduction in the number of machines required to perform breast radiography and biopsy.

The technical aim and objects specified are substantially achieved by a radiography apparatus as claimed in claim 1 and one or more of the dependent claims.

### BRIEF DESCRIPTION OF THE DRAWINGS

Further characteristics and advantages of the present invention will become clearer from the indicative and therefore non-limiting description of a preferred but not exclusive embodiment of a radiography apparatus as illustrated in the accompanying drawings, in which:

FIG. 1 illustrates a first embodiment of a radiography apparatus according to the present invention, in a schematic lateral view and in a first operational configuration;

FIG. 2 illustrates a detail of the apparatus of FIG. 1 in a schematic rear view;

FIG. 3 illustrates the apparatus of FIG. 1 in a schematic lateral view, in a further operational configuration;

FIG. 4 illustrates the apparatus of FIG. 1 in a schematic lateral view and in a second operational configuration;

FIG. 5 illustrates a second embodiment of a radiography apparatus according to the present invention in a schematic lateral view and in a first operational configuration;

FIG. 6 illustrates a detail of the apparatus of FIG. 5 in a schematic rear view with some parts removed in order to better highlight others;

FIG. 7 illustrates the apparatus of FIG. 5 in a schematic lateral view and in a second operational configuration;

FIG. 8 illustrates a third embodiment of a radiography apparatus according to the present invention, in a schematic lateral view and in a first operational configuration;

FIG. 9 illustrates the equipment of FIG. 8 in a schematic rear view;

FIG. 10 illustrates the apparatus of FIG. 8 in a schematic lateral view in an intermediate configuration between the first and a second operational configuration;

FIG. 11 illustrates the apparatus of FIG. 8 in a schematic lateral view in a second operational configuration.

### DETAILED DESCRIPTION OF THE INVENTION

In accordance with the accompanying drawings, the number 1 indicates a radiography apparatus according to the present invention.

The apparatus 1 comprises a supporting column or stand 2 having a main body 2a with main direction of development D.

The apparatus 1 comprises a base 3 which rests on the ground to which the column 2 is secured as will be better described below.

The apparatus 1 comprises a radiography assembly 4 provided with an emitter 5 of radiography rays, for example X rays, and a corresponding detector 6, analogue or digital, for acquisition of the image obtained by means of said rays.

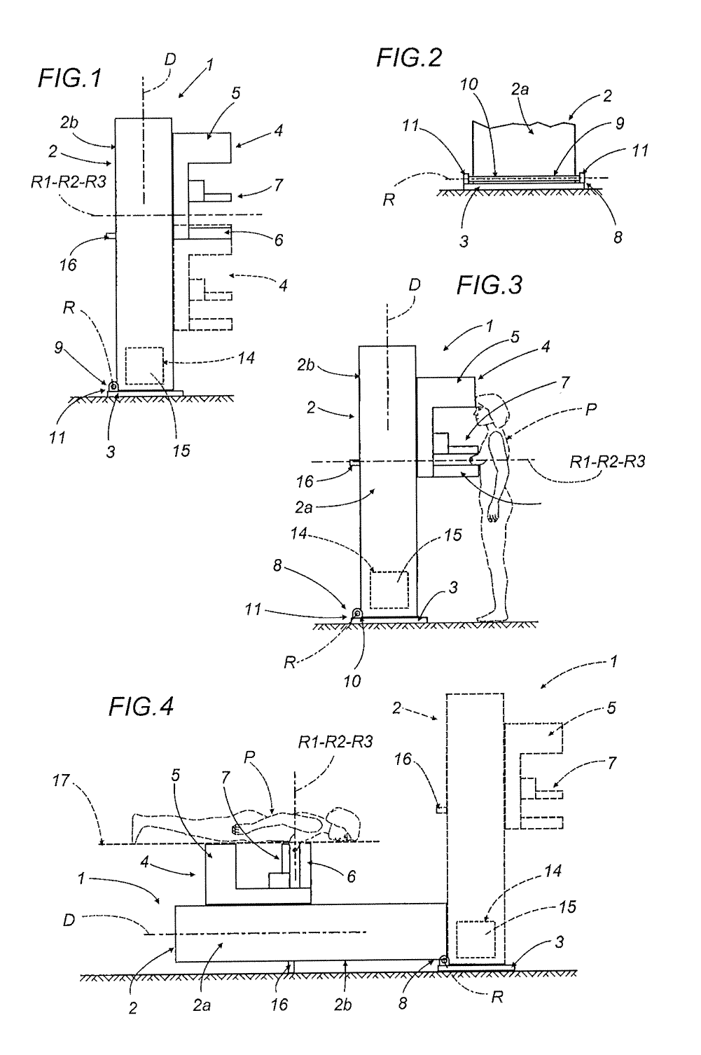

The radiography assembly 4 is secured in a sliding manner to the column 2 and moves along the same between a first operational position, illustrated in FIG. 1, and a second operational position, illustrated by the broken line in FIG. 1, according, for example, to the height of a patient P undergoing the radiographic examination.

The apparatus 1 comprises a system 7 for positioning the breast to be examined and maintaining it in position between said emitter 5 and detector 6.

According to the illustrations of FIGS. 2 and 6 in particular, the apparatus 1 comprises coupling means 8 operationally active between the base 3 and the column 2.

The coupling means 8, which will be better illustrated below, reciprocally secure the base 3 and the column 2 so that the column 2 is mobile with respect to the base 3.

Advantageously, the column 2 moves with respect to the base 3 between a first operational position, below also indicated as raised position, and a second operational position, below also indicated as lowered position.

The column 2 defines with the base 3 an angle “ ” which is between 80° sexagesimal, substantially corresponding to the first operational position, and 200° sexagesimal, substantially corresponding to the second operational position, according to the relative position of the column 2 with respect to the base 3.

For the sake of simplicity, the movement of the column from the raised position to the lowered position will also be indicated by the term “tipping”.

Advantageously, the column 2 can assume any one of the intermediate positions between the first and the second operational position.

It can be noted that in a first preferred operating position, suitable for example for performing mammography, illustrated in FIGS. 1, 3, 5 and 8, the main direction of development D is substantially vertical.

In a second preferred operating position, suitable for example for performing biopsies, illustrated in FIGS. 4, 7 and 11, the main direction of development D is substantially horizontal.

In the preferred embodiment illustrated in FIGS. 1, 2, 3 and 4, the coupling means 8 comprise a hinge 9 having rotation axis R.

The axis R is transverse with respect to the main direction of development D, and in particular, with reference to the example illustrated, orthogonal to it.

In this way, the stand 2 passes from the raised configuration to the lowered configuration by a rotatory movement about the axis R.

In practice, the coupling means 8 comprise, in the preferred embodiment illustrated in FIGS. 1, 2, 3 and 4, a first engagement element 10 combined with the column 2 and a corresponding second engagement element 11 combined with the base 3.

The first and the second engagement element 10, 11 are reciprocally secured to define said hinge 9.

In other words, the engagement elements 10, 11 cooperate to define the movement of the column 2 with respect to the base 3 in transition of the column from the raised position to the lowered position and vice versa.

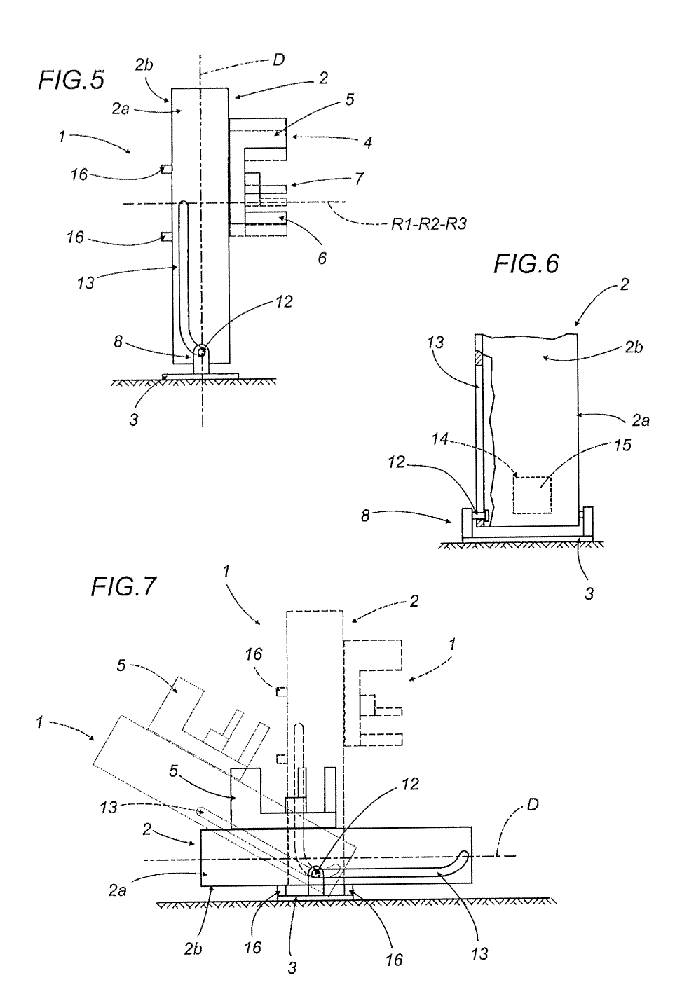

According to the illustrations of FIGS. 5, 6 and 7, the coupling means 8 comprise a pair of pins 12, integral with the column 2.

The pins 12 are arranged on opposite sides of the column 2 with respect to the main body 2a.

The coupling means 8 also comprise a pair of guides 13 combined with the column 2.

In the preferred embodiment illustrated, the guides 13 are obtained on a lateral surface of the main body 2a.

The pins 12 are engaged in a sliding manner in the guides 13:

the movement of the column 2 is guided by the pins 12 by means of the guides 13.

The guides 13 remain engaged in the pins 12 during transition of the column 2 from the raised to the lowered position and vice versa, thus defining the movement of the column 2.

The movement of the column 2, in transition of the column 2 from the raised position, illustrated in FIG. 5, to the lowered position, illustrated in FIG. 7, is therefore a roto-translatory motion.

Advantageously, in order to facilitate tipping of the column 2 and also re-positioning in the raised position from the lowered position, the apparatus 1 comprises a system for movement of the column 2, schematised in a block 14.

Preferably the system 14 comprises a motor or a motor assembly, schematised in a corresponding block 15, for raising and lowering the column 2.

Advantageously, the movement system 14 can be operated by a user not illustrated to recline and raise the column 2.

In the preferred embodiments illustrated, the emitter 5 moves about a rotation axis R1 transverse to the main direction of development D of the column 2.

Advantageously, also the detector 6 moves about a rotation axis R2.

According to the illustrations, preferably also the system 7 for positioning the breast and maintaining it in position moves about a rotation axis R3. Preferably, according to the illustrations, the rotation axes R1, R2 and R3 coincide.

With particular reference to FIGS. 4 and 7, it can be seen that the apparatus 1 comprises feet 16 for resting on the ground which extend from a rear wall 2b of the column 2.

The feet 16 ensure greater stability for the apparatus 1 in the horizontal position.

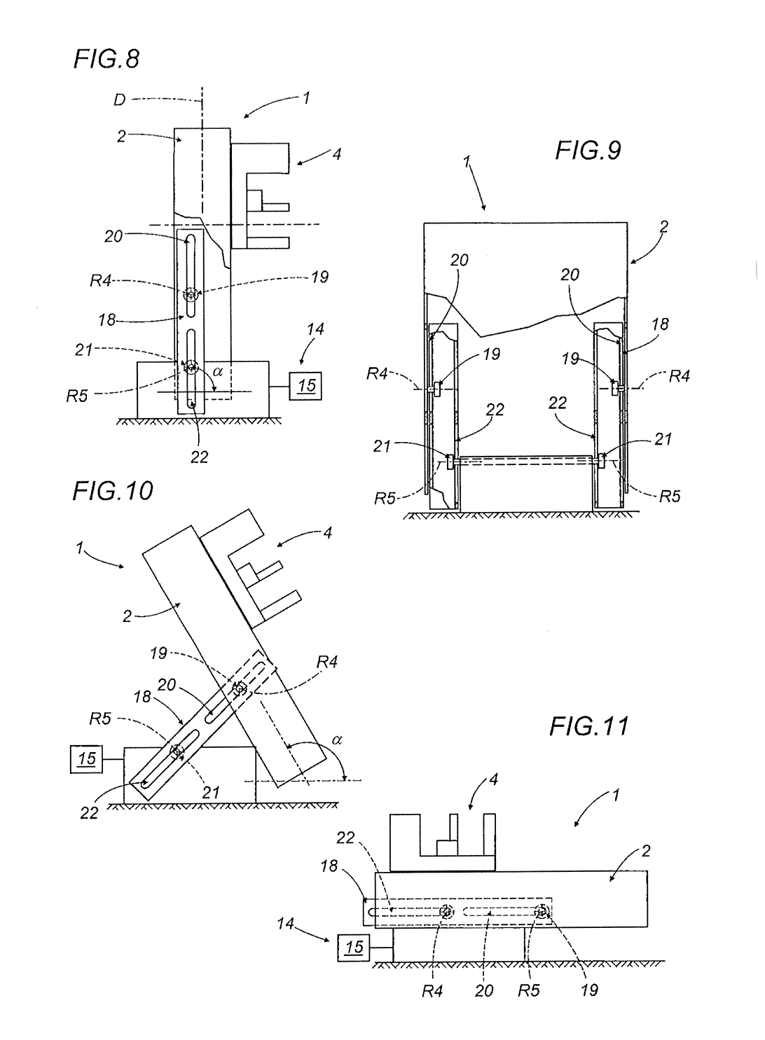

A third embodiment of the apparatus 1 is illustrated by way of example in the FIGS. 8 to 11.

The column 2 is secured at the base 3 by means of a pair of members 18.

In particular, the column 2 is secured to each member 18 by means of a respective pin 19 combined with the column 2.

The members 18 are provided with a slot 20, which develops along said member 18, inside which the corresponding pin 19 is secured so as to slide and rotate.

The pins 19 define a rotation axis R4 about which the column 2 rotates.

Each member 18 is therefore secured to the base 3.

In particular, in the embodiment illustrated, a pair of second pins 21 extends from the base 3, preferably from opposite parts thereof.

The cited members 18 are provided with a second slot 22 in which the corresponding pin 21 is engaged.

The pins 21 are secured to the respective slot 22 so as to rotate and slide therein.

In other words, the pins 21 define a rotation axis R5 around which the members 18 revolve.

In this way, in transition from the raised position to the lowered position (or vice versa) the column 2 performs a rotation about the axis R4.

The pins 19 slide along the respective slot 20 while the members 18 perform a rotation about the axis R5.

The members 18 are free to slide with respect to the pin 19 by means of the slot 20, for example according to a translatory movement.

In a substantially analogous manner, the members 18 rotate about the axis R5 and slide with respect to the pins 21 along the slot 22.

Advantageously, also in this embodiment, a motorised system 14 for movement of the column 2 is preferably provided.

In use, for example when performing mammography, for greater comfort of the patient P the column 2 can be maintained vertical, as illustrated for example in FIG. 3.

The breast is immobilised and compressed in the system 7, and a first projection is performed by emission of rays from the emitter 5 which are captured by the detector 6.

After completion of the first projection, the breast is released, the emitter 5, the detector 6 and the system 7 are rotated about the rotation axis, in a configuration not illustrated, the breast is compressed again and a second projection is then performed.

In the case of a biopsy, it is preferable, as mentioned, to arrange the breast of the patient P in a pendulous position corresponding to the radiography.

The column 2 is then reclined and set to a substantially horizontal position, as in FIG. 4 for example. In this way the system 7 can be reached from an upper position; the patient can be laid on an appropriate support 17 with the breast falling between the emitter 5 and the detector 6.

The breast is compressed in the system 7 which remains stationary with respect to the detector 6 and undergoes a first radiography. Then the emitter 5 only is rotated about the respective rotation axis R1, to a position not illustrated, to perform at least one second radiography and locate the mass in order to withdraw a sample thereof.

In the case of tomosynthesis, once the breast has been immobilised, the emitter 5 and detector 6 rotate about the same, in a known manner not described and illustrated, to obtain a 3D image of the breast.

The invention as described offers important advantages.

The apparatus 1 allows the performance of radiological breast examinations both with the patient standing and examination with the breast in a pendulous position, since the patient can be positioned horizontally.

Said functions allow laboratory optimisation as they no longer require two different machines, for example, for mammography and biopsy.

The performance of mammography followed by biopsy using the same apparatus greatly reduces the risk of loss of the references for the location of any suspicious masses and loss of centring of the lesion when passing from one operation to the next one. The risk of the patient fainting is also substantially reduced. The quality of the image is constant in the two positions since the same instrument is used, thus allowing full utilisation of hospital spaces.

1. Radiography apparatus for mammography comprising: a supporting column or stand (2) having a main direction of development (D); a ground supporting base (3) to which said column (2) is secured; a radiography assembly (4) comprising an emitter (5) of rays for radiography, a corresponding detector (6) for acquisition of the image obtained by means of said rays and a system (7) for positioning and maintaining in position a breast to be X-rayed, operationally active between said emitter (5) and detector (6); said radiography assembly (4) being secured in a sliding manner to said column (2) and moving along the same between a first operating position and a second operating position; said column (2) being mobile with respect to said base (3) between a first raised operational position and a second lowered operational position; the apparatus furthermore comprising coupling means (8) operationally active between said base (3) and said column (2) defining at least one rotation axis (R, R1, R2, R3, R4, R5) for said column (2), said column rotating around said axis (R1, R2, R3, R4, R5) in the transition between said first operational position and said second operational position; said coupling means (8) comprising first engagement means (10, 13, 19) combined with said column (2) and second engagement means (11, 12, 21) combined with said base (3), said first engagement means (10, 13, 19) and said second engagement means (11, 12, 21) cooperating to define the movement of said column (2) with respect to said base (3) in the transition of said column (2) from said raised first operational position to said second operational position and vice versa; said movement being a roto-translatory motion.

2. Apparatus as claimed in claim 1, wherein said main direction of development (D) is substantially vertical corresponding to said first operational position and is substantially horizontal corresponding to said second operational position.

3. Apparatus as claimed in claim 1, wherein said coupling means (8) comprise a hinge (9) with axis (R) transverse with respect to said main direction of development (D).

4. Apparatus as claimed in claim 1, wherein said coupling means (8) comprise at least one member (18) operationally active between said column (2) and said base (3), said first and said second engagement means (19, 21) comprising respective pins (19, 21) engaged in a sliding manner in said members (18).

5. Apparatus as claimed in claim 1, and comprising means (14, 15) for movement of said column (2) operationally active on said column (2) and which can be operated by a user to move said column (2) between said first operational position and said second operational position.

6. Apparatus as claimed in claim 5, wherein said movement means (14, 15) comprise a motor (15) for raising and lowering said column (2).

7. Apparatus as claimed in claim 1, wherein said emitter (5) moves around a rotation axis (R1) transverse to said main direction of development (D).

8. Apparatus as claimed in claim 1, wherein said detector (6) moves around a rotation axis (R2) transverse to said main direction of development (D).

9. Apparatus as claimed in claim 1, wherein said positioning system (7) moves around a rotation axis (R3) transverse to said main direction of development (D).

10. Apparatus as claimed in claim 7, wherein said rotation axis (R1) of said emitter (5), said rotation axis (R2) of said detector (6) and said rotation axis (R3) of said positioning system (7) coincide.
